(19) United States
(12) Patent Application Publication
(10) Pub. No.: US 2013/0158613 A1
(43) Pub. Date: Jun. 20, 2013

(54) CARDIOVERTERS FOR ELMINATING ATRIAL FIBRILLATION

(71) Applicant: Peter Osypka, Grenzach-Wyhlen (DE)

(72) Inventor: Peter Osypka, Grenzach-Wyhlen (DE)

(21) Appl. No.: 13/816,345

(22) PCT Filed: Jul. 28, 2011
(86) PCT No.: PCT/EP2011/003789

§ 371 Date: Feb. 11, 2013

(30) Foreign Application Priority Data
Aug. 10, 2010 (DE) 20 2010 01 1244.3

(51) Int. Cl. A61N 1/39

(52) U.S. Cl. CPC A61N 1/39
USPC 607/5

(57) ABSTRACT

The invention relates to two independently operating electrical cardioverters, each having an integrated stimulator for heart therapy. The cardioverters and stimulators have two separate final stages, which can deliver shock pulses or stimulation pulses at a time offset from each other, but can also be controlled by the ventricular ECG and synchronized accordingly.

### BACKGROUND

The invention relates to a cardioverter with an integrated stimulator for therapy of the heart. Here, the invention relates to two independently operating electrical systems for therapy of the heart, formed of a cardioverter and an integrated stimulator. Here, cardioverter and stimulator each have two separate final stages, which can each emit an energy pulse at a time offset from one another, or which can synchronously be synchronized by the ventricle EKG.

Postoperative atrial fibrillation occurs relatively frequently and is one of the most important reasons for postoperative morbidity. Overall, atrial fibrillation after cardiac surgery appears to have increased in recent years; this is explained by an aging patient collective. References relating to postoperative atrial fibrillation in the case of patients with preexisting sinus rhythm showed an average occurrence in 30%-40% after bypass surgery on the heart. Atrial fibrillation leads to a fast transmission of the excitement to the ventricle, and so this can lead to acute hemodynamic instability. Electrical cardioversion is a non-medicinal and very effective method for restoring the sinus rhythm; however, it does require brief anesthesia. However, particularly in the case of patients after bypass surgery, this brief aesthesia can increase the existing neuronal problems (vigilance) as a result of the recently endured heart operation, which may lead to a lengthened waking-up phase or even necessitate renewed intubation with machine ventilation. Anticoagulation in postoperative patients with atrial fibrillation constitutes a further problem. If the arrhythmia continues for more than 24 hours, anticoagulation is required in order to reduce the thrombus formation with the risk of a cerebrovascular accident. All these factors lead to complicated postoperative progress in the case of patients after bypass surgery, which is reflected in a hospital stay which is lengthened by approximately five days, incurring increased costs.

Until now, atrial fibrillation after a heart operation has been eliminated by an external electrical energy pulses by means of a defibrillator, applied by applying or adhering large-area electrodes to the chest of the patient. For this, it is mandatory for an ultrasound examination of the left auricle of the heart to be performed on the patient in advance, and for anesthesia to be required during the cardioversion.

US 2002/0072775 A1 and US 2006/0217769 A1 respectively disclose an implantable instrument for stimulation and defibrillation; however, said instrument is not suitable for direct postoperative application because it has to be implanted for use.

### SUMMARY

Thus, there is the object of developing a cardioverter which enables a direct, reliable and as pain-free as possible elimination of the atrial fibrillation of the heart.

This object is achieved by one or more of the means and features of the invention. Advantageous embodiments or developments are described below and in the claims.

As a result of the cardioversion being performed simultaneously at the two atria in an intracardial and local fashion, but with separate energy pulses, the required electrical energy emission for cardioversion reduces substantially. This opens up the possibility of directly eliminating the atrial fibrillation without anesthetics for the patient. The cardioverter can naturally also be operated without stimulator.

A particularly advantageous embodiment of the invention, which is significant enough to be protected in its own right, can consist of the fact that the cardioverter with the stimulator is subdivided into two parts, that the two parts, respectively separately, have electrodes, in particular bipolar electrodes, which can be attached to the two atria of the heart and that, in the usage position, the cardioversion can be performed simultaneously at the two atria in an intracardial and local fashion, but with separate energy pulses. The cardioverter and the stimulator thereof each have two separate final stages, which are connected to separate bipolar electrodes for attachment to the outer sides of the atria of the heart. As a result of this, the already mentioned direct elimination of the atrial fibrillation without anesthetics is made possible in a simple manner.

As a result of the subdivision into two parts, the cardioverter has two outputs and bipolar epicardial electrodes originating therefrom, i.e. it has a simple design.

Here, means are expediently provided for ensuring that the emitted shock pulses for defibrillation or cardioversion of the two atria of the heart are offset in time. Although these shock pulses can be applied simultaneously, they can also be applied with a time offset.

Expedient magnitudes for the respective energy pulses for the cardioversion are specified below.

The sought-after objective is solved particularly well by on embodiment, namely according to which the two electrodes in the atria and the outputs of the cardioverters are or can be connected in such a way that the energy pulses for the cardioversion are effective, with appropriate polarity, between the two atria of the heart.

A further option can consist of the outputs of the cardioverters being or being able to be connected in such a way that the energy pulses for the cardioversion for the two atria of the heart are or can be transmitted telemetrically.

A further option or embodiment can provide for the outputs of the cardioverters or of the two parts of the cardioverter to be or to be able to be connected in such a way that the energy pulses for the cardioversion can be emitted between atrium and an external indifferent electrode.

Furthermore, it is possible for the energy pulses for the cardioversion to be emitted between atrium and an indifferent electrode in the esophagus.

An expedient embodiment of the invention can provide for provision to be made for a further bipolar sensing electrode. Thus, the cardiac activity can be monitored by such a sensing electrode.

The whole system can be triggered or synchronized by the ventricle EKG, in particular by means of the bipolar sensing electrode.

Further details, features and advantages of the invention can be gathered from the following part of the description, in which the invention is explained in more detail on the basis of the drawing and the exemplary embodiment.

### BRIEF DESCRIPTION OF THE DRAWINGS

FIG. 1 shows a schematic illustration of a cardioverter/stimulator (subdivided into (1) and (2)), respectively separated for the two atria with the corresponding electrodes (11) and (12) at the heart.

### DETAILED DESCRIPTION OF THE PREFERRED EMBODIMENT

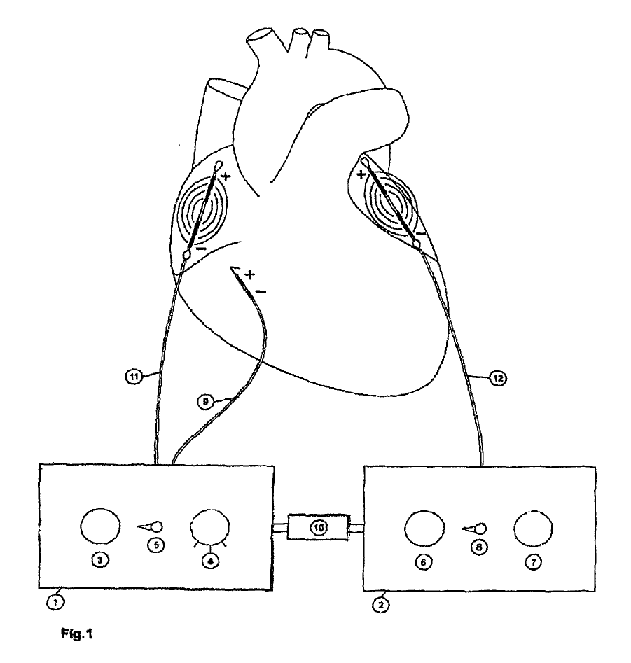

FIG. 1 shows two bipolar electrodes 11 and 12, which are attached to the two atria of the heart and which are connected to a cardioverter which simultaneously also contains a stimulator (subdivided into (1) and (2)). The cardioverter can emit two bi-phase energy pulses synchronously and simultaneously or with a delay of, for example, 0-500 ms which can be set by a control device 10. The two outputs of the cardioverter have a separate isolated design such that the output signals cannot influence one another in any way. The energy to be emitted by each output can be set in a range of between 1 and 20 joules, for example, by the setting devices 3 and 6; however, larger energy emissions are also possible. Stimulation amplitude, stimulation frequency and stimulation sensitivity can be set by setting devices 4 and 7, wherein these can be designed separately or as one unit.

The atria of the heart can be electrically stimulated after each cardioversion, and also during the atrial fibrillation-free state of the heart. To this end, the cardioverter is switched by the switching devices 5 and 8 and the stimulator likewise contained therein is activated. According to requirements, the stimulator is able to stimulate the two atria and ventricles of the heart in a conventional fashion. The whole system can be triggered or synchronized by the ventricle EKG (QRS complex). To this end, a further bipolar sensing electrode 9 is illustrated in FIG. 1 in an exemplary fashion.

The two parts of the cardioverter and hence the two bipolar electrodes on the atria can also be connected in such a way that the energy emission occurs between the two atria. Moreover, as a result of the separate isolated design, the right atrium can, for example, be stimulated while the left atrium is cardioverted. Here, particular care will be taken in respect of the stimulation of the atria of the heart in order to avoid a possible recurrence of the atrial fibrillation. Should therapy of the atrial fibrillation in this manner not result in lasting success and the patient has to subject himself to e.g. RF ablation, the energy application can also be continued telemetrically over a relatively long period of time by means of suitable coils (not illustrated here). The telemetric transmission of energy and information is prior art and will not be explained in any more detail here. The position, the type of embodiment and the attachment of the coils can, depending on the anatomy, be implanted on different parts in the human body such that a subsequent explanation can easily be carried out. The cardioverter and the stimulator must also be equipped with corresponding transmission coils.

By way of example, the cardioverter with battery can be positioned externally on the belt of the patient for this period of time.

A further type of cardioversion can be brought about by virtue of the energy pulses being applied against a common neutral external electrode (not illustrated here).

A further type of cardioversion can be brought about by virtue of the energy pulses being applied against a common neutral electrode which, as a catheter with a corresponding large-area electrode, is situated in the esophagus (not illustrated here).

1. Two independently operating electrical systems for therapy of the heart, including a cardioverter and an integrated stimulator, characterized in that cardioverter and the stimulator each have two separate final stages, which emit an energy pulse in a respectively independently adjustable fashion at a time offset from one another, or which can synchronously be synchronized accordingly by a ventricle EKG.

2. A cardioverter with an integrated stimulator for therapy of the heart, characterized in that the cardioverter with the stimulator is subdivided into two parts (1, 2), in that the two parts, respectively separately, have electrodes (11 and 12) which can be attached to two atria of the heart and in that, in a usage position, the cardioversion can be performed simultaneously at the two atria in an intracardial and local fashion, but with separate energy pulses.

3. The cardioverter and stimulator as claimed in claim 2, characterized in that the cardioverter and the stimulator thereof each have two separate final stages (1, 2), which are connected to separate bipolar electrodes (11, 12) for attachment to outer sides of the atria of the heart.

4. The cardioverter with stimulator as claimed in one of claims 1 to 3, characterized in that the cardioverter, as a result of being subdivided into two parts (1 and 2), has two outputs and bipolar electrodes (11 and 12) originating therefrom.

5. The cardioverter with stimulator as claimed in one of claims 1 to 4, characterized in that means are provided for ensuring that emitted shock pulses for defibrillation of the two atria of the heart are offset in time.

6. The cardioverter with stimulator as claimed in one of claims 1 to 5, characterized in that a setting device (10) is provided for the time delay of the energy pulses or shock pulses from the two parts (1 and 2) of the cardioverter with the stimulator.

7. The cardioverter and stimulator as claimed in one of claims 1 to 6, characterized in that the respective energy pulses for the cardioversion can be set between 0 and 20 joules.

8. The cardioverter and stimulator as claimed in one of claims 1 to 7, characterized in that the respective energy pulses for the stimulator can be set between 0 and 30 volts and with a pulse width of between 0.5 ms and 20 ms.

9. The cardioverter and stimulator as claimed in one of claims 1 to 8, characterized in that the energy emissions for a left atrium can be set to have a delay of between 0 and 500 ms compared to a right atrium.

10. The cardioverter and stimulator as claimed in one of claims 1 to 9, characterized in that the two bipolar electrodes (11, 12) at the atria and outputs of the cardioverters (1, 2) are or can be connected in such a way that the energy pulses for the cardioversion can be performed, with appropriate polarity, simultaneously but with separate energy pulses at the two atria.

11. The cardioverter as claimed in one of the preceding claims, characterized in that the two epicardial electrodes (11 and 12) at the atria and the outputs of the cardioverters are or can be connected in such a way that the energy pulses for the cardioversion are effective, with appropriate polarity, between the two atria of the heart.

12. The cardioverter and stimulator as claimed in one of claims 1 to 11, characterized in that the outputs of the cardioverters (1, 2) are or can be connected in such a way that the energy pulses for the cardioversion for the two atria of the heart are transmitted telemetrically.

13. The cardioverter and stimulator as claimed in one of claims 1 to 12, characterized in that the outputs of the cardioverters (1, 2) are or can be connected in such a way that the energy pulses for the cardioversion can be emitted between atrium and an external neutral electrode.

14. The cardioverter and stimulator as claimed in one of claims 1 to 13, characterized in that the energy pulses for the cardioversion can be emitted between atrium and a neutral electrode in the esophagus.

15. The converter and stimulator as claimed in one of claims 1 to 14, characterized in that provision is made for a further bipolar sensing electrode (9), which preferably controls or synchronizes the progress of the cardioversion.
